(19) United States
(12) Patent Application Publication
(10) Pub. No.: US 2026/0321368 A1
(43) Pub. Date: Oct. 15, 2026

(54) INFORMATION PROCESSING ASSESSMENT BOOTH

(71) Applicant: MAHER RATIB ALDERI, Amman (JO)

(72) Inventor: Noraldin Maher Ratib ALDERI, Amman (JO)

(21) Appl. No.: 19/483,340

(22) PCT Filed: Apr. 16, 2024
(86) PCT No.: PCT/IB2024/053692

§ 371 Date: Nov. 11, 2025

Related U.S. Application Data
(60) Provisional application No. 63/465,708, filed on May 11, 2023.

(51) Int. Cl. G16H 10/40; A61B 5/00; A61B 5/16

(52) U.S. Cl. CPC G16H 10/40; A61B 5/168; A61B 5/6888

(57) ABSTRACT

An information processing assessment booth may be provided with a base panel, having a trapezoidal surface defined in a first plane with a front edge parallel to a back edge, and first and second side edges disposed at a first angle between the front edge and the back edge; a first side panel attached to the first side edge of the base panel, extending perpendicularly to the first plane; a second side panel attached to the second side edge of the base panel, extending perpendicularly to the first plane; a roof panel, connected between the first side panel and the second side panel in a second plane that intersects the first plane; and a divider, connected between the first side panel, the second side panel, the roof panel, and the base panel, extending perpendicularly from the base panel and including a video display device and a video recording device.

### TECHNICAL FIELD

The present disclosure relates to medical testing device. More particularly, the present disclosure relates to a device for testing the informational processing ability of assessed individuals.

### BACKGROUND

When assessing the information processing capabilities of an assessed individual, distractions in the environment can affect the assessment. This is particularly true when attempting to assess individuals with (or suspected of having) autism, attention deficit disorder, or who are very young.

### SUMMARY

A testing booth to reduce outside stimuli competing against stimuli measured by a machine learning algorithm to aid in the diagnosis of information processing deficiencies in assessed individual and prophylaxis against the dangers of misdiagnosis inherent to traditional assessment schemes is provided. Advantageously, the system is able to assess the information processing ability of children as the assessed individuals, including children under the age of four (e.g., between the ages of 4 and 48 months), to assess whether these assessed individuals possess intact information processing capabilities and to aid in the diagnosis and treatment of those individuals who do not possess intact information processing capabilities.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIGS. 1A-1D illustrate several views of a testing booth, according to aspects of the present disclosure.

FIGS. 2A-2P illustrate several views of a testing booth, according to aspects of the present disclosure.

FIGS. 3A-3E illustrate several views of components of a testing booth, according to aspects of the present disclosure.

FIGS. 4A and 4B illustrate additional configurations of a testing booth, according to aspects of the present disclosure.

FIG. 5 is a flowchart of an example method for using the testing booth, according to aspects of the present disclosure.

FIG. 6 is an example computing device, as may be used with a testing booth, according to aspects of the present disclosure.

FIG. 7 illustrates a flow of operation of the machine learning algorithm and programs used in the testing booth, according to aspects of the present disclosure.

### DETAILED DESCRIPTION

The present disclosure provides a system that comprises a booth structure that houses a computing device running an algorithm to assess information processing of assessed individuals. Advantageously, the system is able to assess the information processing ability of children as the assessed individuals, including children under the age of four (e.g., between the ages of 4 and 48 months), to assess whether these assessed individuals possess intact information processing capabilities and to aid in the diagnosis and treatment of those individuals who do not possess intact information processing capabilities. As used herein, information processing capabilities refers to the way that the assessed individual mentally receives, interprets, and remembers information. The present disclosure therefore provides an automated assessment system devised to assist in predicting information processing in various assessed individuals. This system avoids relying on the compliance or the ability to speak in the assessed individual, which many traditional tests rely on. Instead, the system examines the reactions of the assessed individual video stimuli to assess the information processing capabilities of the assessed individual.

To improve the quality of data collection, improve compliance in the assessed individual, and aid in deployment, the system is provided via an expandable booth to reduce the effect of outside stimuli, while still providing access between the assessed individual and the practitioner or other trusted party. Accordingly, although traditional booths for medical examination often enclose the assessed individual or separate the assessed individual from the practitioner (and other parties), which can lead to stress in children or persons uncomfortable with the examination process that negatively affect data collection, the present booth design offers a reassuring layout that allows for easier contact between the assessed individual and other parties during the assessment, while allowing the parties to remain out of eye contact (when desired).

One problem addressed by the present disclosure is that health practitioners currently cannot differentiate between children with intact information processing capabilities and children with defective information processing capabilities. False positives are not nearly as problematic as false negatives (misdiagnosis as typical). When misdiagnosed with defective information processing (e.g., a False Positive result), children are subjected to complex treatment plans, medications, and low expectations leading those children to acquire intellectual disabilities in the future. The practitioners can easily spot false negatives, but false positives are more difficult to spot early, making the present disclosure a novel contribution to diagnosing mental ability and development.

The present disclosure therefore provides an additional tool for health professionals to assess children's information processing with high precision to diagnose mental ability and intellectual disabilities in those children. The present disclosure provides a detailed report of a child's performance with an assessment of their information processing and risk group. The present disclosure also offers additional information to help the practitioner provide a better diagnosis, which include one or more of heart rate changes, gazing, fixation, and emotional changes in response to a visual stimulus. When the present disclosure is used in combination with the current tools and the practitioner's knowledge, a more precise prediction of the child's mental ability is provided in a few minutes.

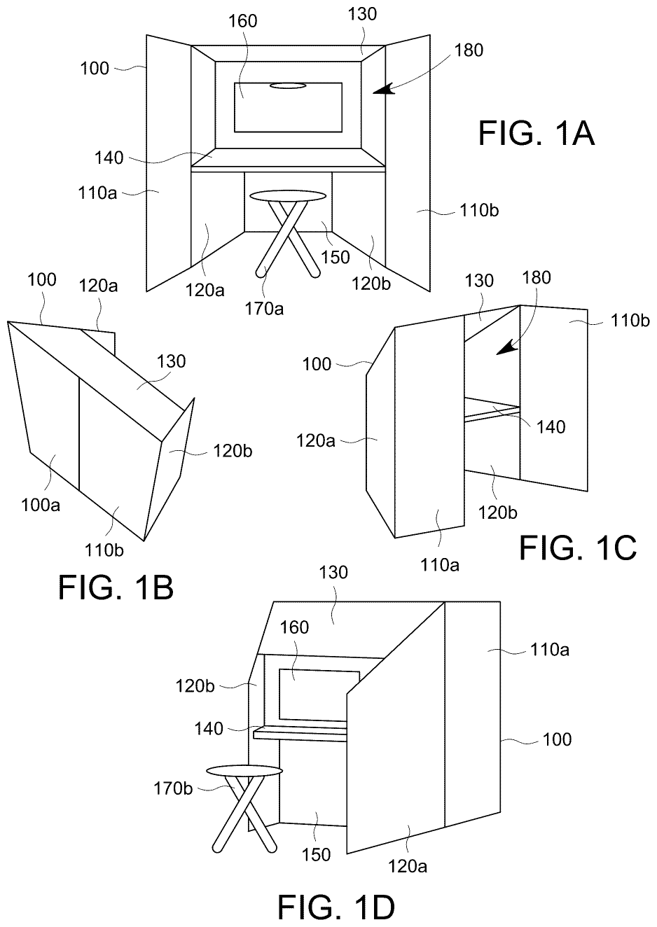

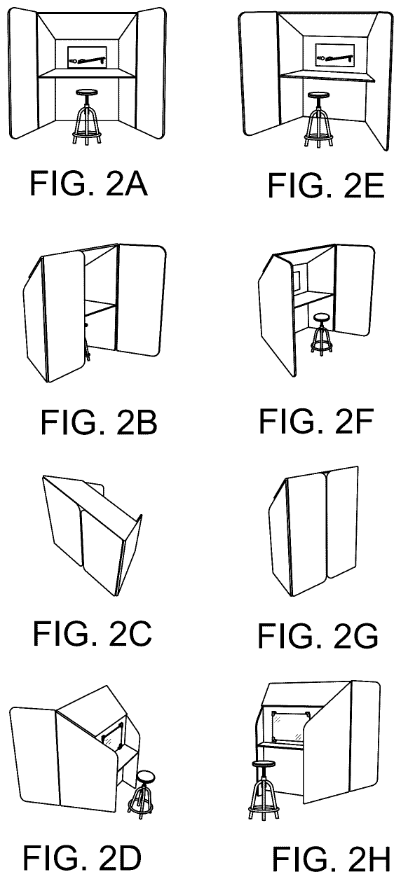

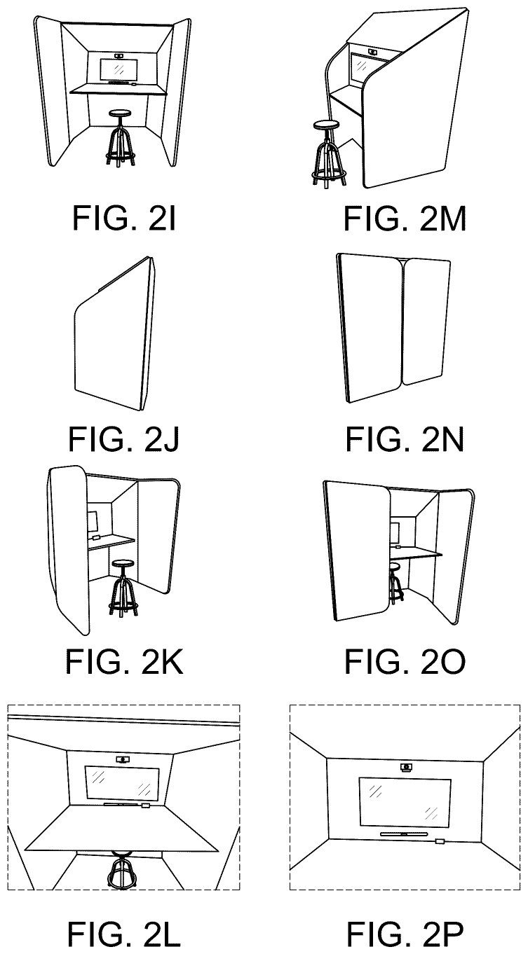

FIGS. 1A-1D and FIGS. 2A-2P illustrate several views of a testing booth, according to aspects of the present disclosure.

As shown, the booth 100 includes several panels that can be arranged in different configurations. The booth 100 is designed to incorporate an acoustic environment to minimize the noise and distraction of the assessed individual. Two front panels 110a-b (generally or collectively, front panels 110) act as separators on the sides of a seating area for the assessed individual (e.g., where a first chair 170a is located) to shield the assessed individual from any distractions when deployed, and may be folded inward to close off the booth 100 to save space, protect/store away items in the internal compartment of the booth 100, and encourage novelty in the assessed individual when the booth 100 is opened. This design proves helpful during the testing of assessed individual in otherwise very distracting environments such as studios, nurseries, and hospitals.

In various aspects, the front panels 110a-b are connected to associated side panels 120a-b (generally of collectively, side panels 120) than angle outward to a seating area for the assessed individual (e.g., where a first chair 170a is shown) from a seating area for a practitioner (e.g., where a second chair 170b is shown). This angled design provides for greater stability of the booth 100, and defines a viewing funnel 180 for the assessed individual to focus inward toward a computing device 160 displaying the video stimuli and collecting the reaction data. In various aspects, the side panels 120 are attached to the front panels 110 via various hinges, joints, thinned sections in a shared material, or the internal and/or external covering of the booth 100. In some embodiments, the front panels 110 may be omitted or may be replaced with extensions of the corresponding side panels 120a-b.

A base panel 140 and a roof panel 130 define a viewing funnel 180 with the interior of the side panels 120. In various aspects, the base panel 140 is positioned at a predefined distance from a floor that the first side panel 120a and the second side panel 120b rest on. The base panel 140 may be connected to the side panels 120 via slots in the side panels 120, shelves defined on the side panels 120, angle brackets, or the like. The roof panel 130 may be connected to the side panels 120 via the upper edges of the side panels 120, stoppers defined on the side panels 120, angle brackets, or the like.

In various aspects, the roof panel 130 is mounted to the booth 100 at an angle relative to the plane that the base panel 120 is disposed in, such that the plane that the roof panel 130 is disposed in intersects the plane of the base panel 120. In various aspects, the angle of intersection between these planes is substantially equal to the angle at which the side panels 120 are angled inward from the assessed individual seating area towards the assessing individual seating area. Accordingly, the shape of the viewing funnel 180 to direct the assessed individual's gaze towards a video device 160 disposed in a divider 150 that blocks the view from one side of the booth 100 to the other.

The video device 160 may include a video display device and a videorecording device, either as separate devices or as a combined device. The video display device is provided to output video (which may include or exclude sound) to the assessed individual as part of a test for information processing capabilities, and the video recording device captures the reactions of the assessed individual in relation to the videos.

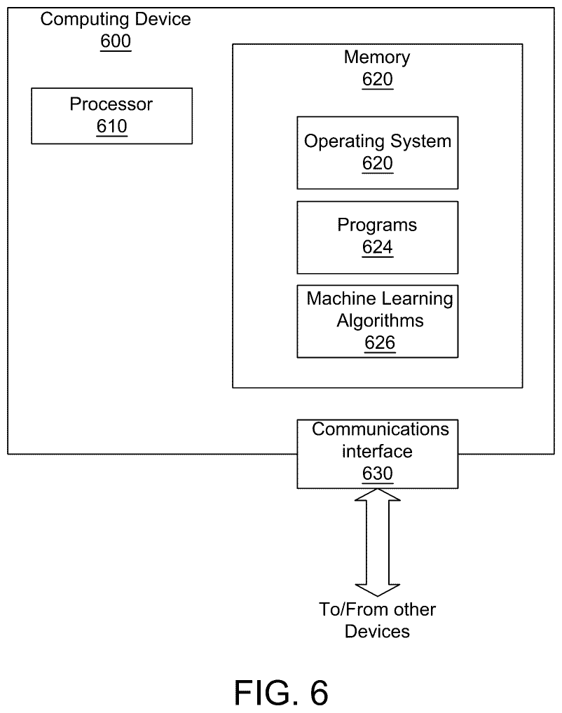

In various aspects, the video device 160 may include, or be in communication with, a computing device, such as is discussed in greater detail in regard to FIG. 6. Additionally or alternatively, other recording devices or programs for analyzing recorded video of the reactions of the assessed individual may be used included, but not limited to: heart rate monitoring, gaze detection, facial expression detection, breathing rate monitoring, brain wave monitoring, skin conductivity, or the like.

In some aspects, the video device 160 include a control panel on an opposite side of the divider 150 to where the video recording device and video display device are provided so that a practitioner can view the video of the captured reactions of the assessed individual, select which videos to output as part of the assessment, control when and how the assessment in provided, or the like.

The unique design and shape of the booth helps assessed individuals with ADHD, autism, and other neuro-cognitive abnormalities to focus with the test and increases the attention span of these assessed individual to last for longer testing that if the tests had been formed in the same environment, but not using the booth 100.

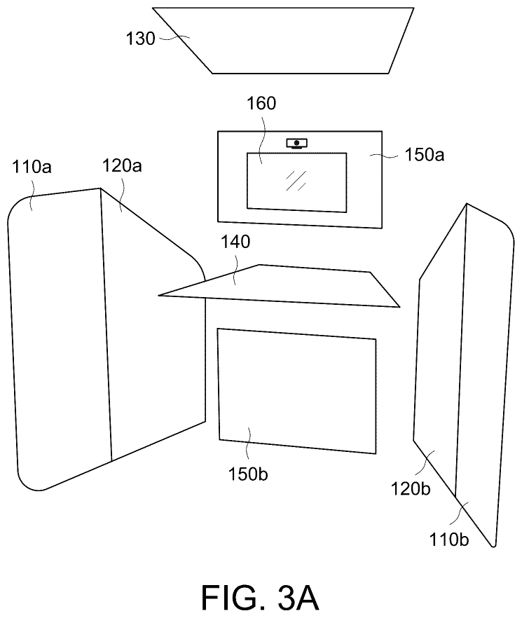

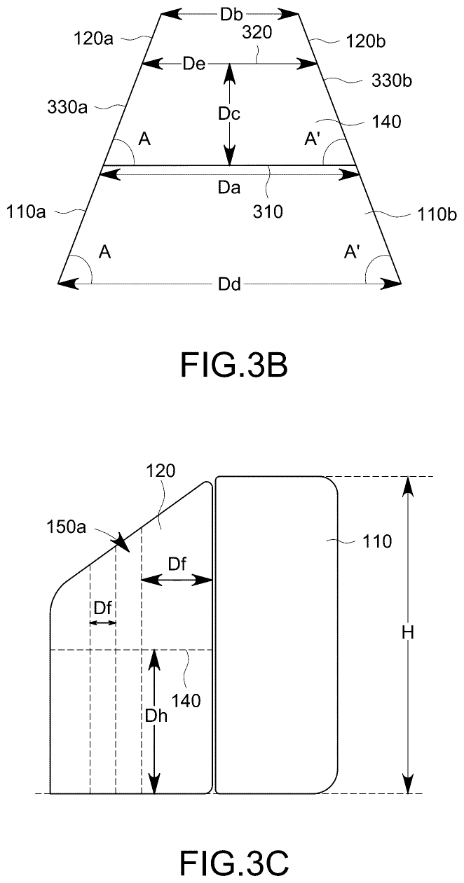

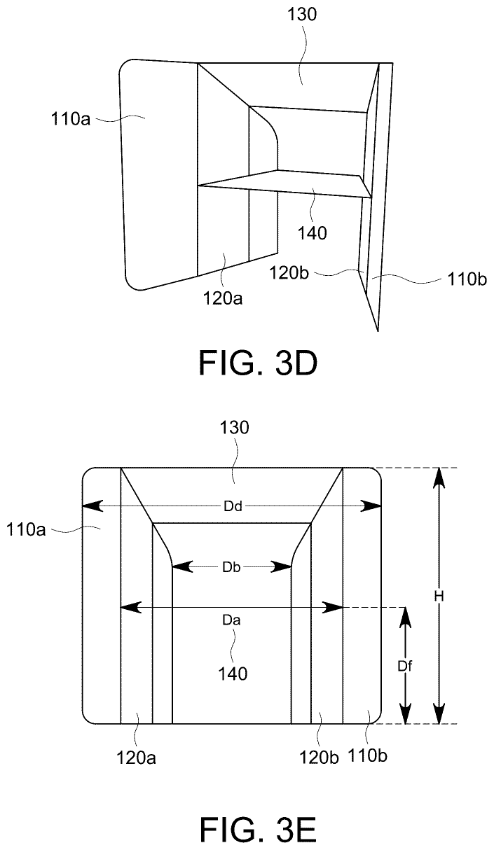

FIGS. 3A-3E illustrate several views of components of a testing booth, according to aspects of the present disclosure. The example booth 100 may be understood as including: a base panel 140, having a trapezoidal surface defined in a first plane with a front edge 310 parallel to a back edge 320, a first side edge 330a disposed at a first angle (A) between the front edge and the back edge, and a second side edge330b disposed at an inverse of the first angle (A′) between the front edge and the back edge; a first side panel 120a attached to the first side edge 330a of the base panel 140, extending perpendicularly to the first plane; a second side panel 120b attached to the second side edge 330b of the base panel, extending perpendicularly to the first plane; a roof panel 130, connected between the first side panel 120a and the second side panel 120b in a second plane that intersects the first plane; and a divider 150, connected between the first side panel 120a, the second side panel 120b, the roof panel 130, and the base panel 140, extending perpendicularly from the base panel 140 and including a video display device and a video recording device. As used herein, the inverse of a given angle may be understood as the given angle at an opposite direction from a reference axis (e.g., X degrees clockwise from an axis and X degrees counterclockwise from the axis).

In various aspects, the divider 150 may be provided in one or multiple parts(e.g., divider 150a and divider 150b) that are included to block the ability for the assessed individual to see to the other side of the booth 100. Accordingly, the video display device and the video recording device are included on first side of the divider 150, facing towards the front edge 310 and the seating area for the assessed individual. In some aspects, the divider 150 includes, on a second side of the divider 150, opposite to the first side, a control device included in or in communication with the computing device driving the video device 160. Accordingly, a practitioner performing the assessment may be out of view of the assessed individual, but remains nearby and able to view the assessed individual and control the assessment. In various embodiments, the height H of the booth 100 is less than or equal to 1.8 meters (m), 1.5 m, 1.2 m, etc., so that, should the assessed individual become upset or scared during the assessment, the practitioner, or any other individual blocked from the view of the assessed individual can easily become visible again (e.g., to reassure a scared child).

As shown in FIG. 3B, the relative distances of the deployed booth 100 may be understood as the distance from one side of the front edge 310 to the other (Da), the distance of the first side panel 120a to the second side panel 120b on the practitioner seating area (Db), the distance between the front edge 310 and the back edge 320 (Dc), the distance between the outstretched edges of the first front panel 110a and the second front panel 110b (Dc), and the distance from one side of the front edge 310 to the other (De). Each of these predefined distances may be configured to the needs of the particular booth 100 and the space in which it will be deployed.

The height H of the booth 100 may be controlled by selecting the height of the side panels 120, and the relative distances of the video device 160 from the assessed individual may be controlled positioning the top divider 150a (or controlling the thickness thereof) relative to the front edge 310 or the back edge 320 of the base panel 140, which may provide more or less funneling of vision or more or less residual space on the seating side for the practitioner (e.g., to place paperwork, keyboard, mouse, etc.). For example, a user or designer can adjust the distance between the top divider 150a and the front edge 310 (Df) to increase or decrease the depth of the viewing funnel 180 and can adjust the distance between the top divider 150a and the back edge (Dg) to increase or decrease the amount of the base panel 140 that is available for use as a table/shelf for the practitioner to use. Similarly, the distance from the floor to the base panel 140 (Dh) may be adjusted to change the amount of leg room or space that is blocked via a bottom divider 150b. Each of these predefined distances may be configured to the needs of the particular booth 100 and the space in which it will be deployed.

In various aspects, the booth 100 includes a first front panel 110a rotatably connected to the first side panel 120a; and a second front panel 110b rotatably connected to the second side panel 120b. In some aspects, the front panels 110 may be rotated to be parallel and (optionally) in contact with the front edge 310 to “close” the booth 110, and each front panel 110 is substantially equal to half the length of the front edge 310. In some aspects, the front panels 110 can be rotated to share the angle A of the side panels 120 to further aid in funneling the vision of the assessed individual towards the video device 160.

In some aspects, the angle at which the upper edges of the side panels 120 are inclined position the roof panel 130 such that the second plane (in which the roof panel 130 is disposed) relative to the first plane (in which the base panel 140 is disposed) is substantially equal to the angle A at which the side panels 120 and angled relative to the front edge 310 so that the second plane intersects the first plane at the same angle A (or A′).

The inner surfaces of the panels (e.g., facing the viewing funnel 180) may be of a matte finish, and of a dark neutral color (e.g., gray, black) to focus the vision of the assessed individual without distraction or reflection inward to a video device 160 included in the top divider 150a. These inner surfaces may also include various sound proofing to further decrease distractions from outside of the booth 100 from competing against stimuli provided by the booth 100 and thereby affecting the reactions of the assessed individual to the provided stimuli.

In various aspects, a computing device is stored within or comprises the top divider 150a and is in communication with video display devices and video recording devices that face towards the assessed individual and various control devices that face towards the practitioner. In various embodiments, the computing device may be in wired or wireless communication with various input and output devices (e.g., a heart rate monitor, a keyboard, a mouse, etc.) and may be networked with various other computing devices (e.g., over the Internet, a local network, etc.). The computing device may locally store, or access a remote computing device that stores (e.g., as a cloud service), some or all of the instructions that when executed by a processor provide a machine learning algorithm to assess an information processing capability of an assessed individual who is recorded reacting to video displayed by the video display device. In various aspects, the recorded reactions can include gaze determinations, facial expression determinations, heart rate determinations, or the like.

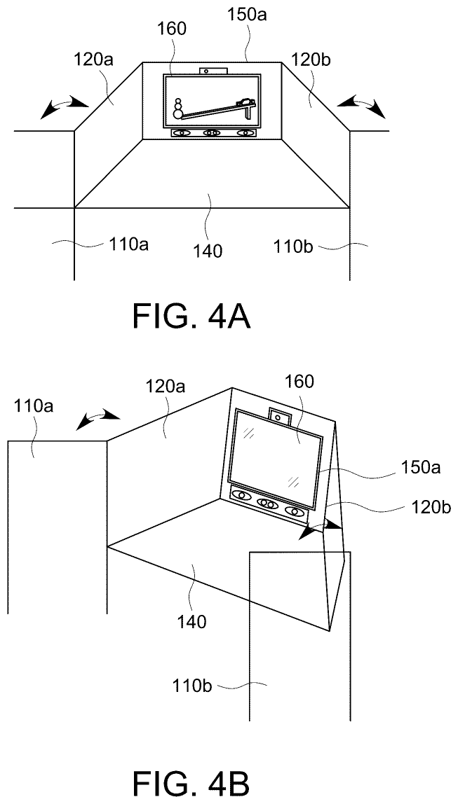

FIGS. 4A and 4B illustrate additional configurations of a testing booth, according to aspects of the present disclosure. As shown with the roof panel 130 removed for purposes of explanation, the front panels 110 may be extended to be parallel with the associated side panels 120, or may be rotated to be non-parallel with the associated side panels 120 when outputting video to account for space availability, to adjust the funneling angle for view of the video, or the like.

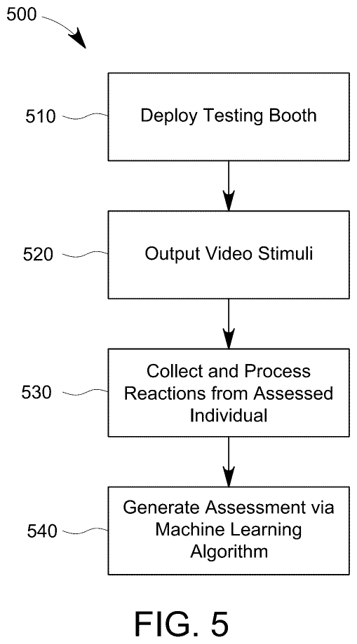

FIG. 5 is a flowchart of an example method 500 for using the testing booth, according to aspects of the present disclosure. Method 500 begins at block 510, where a practitioner deploys the testing booth 100. In various aspects, the practitioner can set the various predefined distances to adjust how high the video device 160 or how deep the video device 160 sits relative to where the assessed individual will sit, what angle the front panels 110 are spread relative to the viewing funnel 180. Once the booth 100 is configured, the practitioner positions the assessed individual in the viewing area to see the video output device of the video device 160 and to be seen by any cameras of other video recording devices, and may connect various non-video sensors (e.g., heart rate monitors, skin galvometers, electroencephalogram sensors, etc.). Then the practitioner, to reduce distraction or providing competing stimulus, and to access the controls for the testing booth 100, sits out of view of the assessed individual in the practitioner seating area. Because the height of the booth is generally less than the average person, the practitioner (or other party out of view behind the booth 100) may easily stand up to reassure the assessed individual if the assessed individual becomes frightened.

At block 520, the practitioner controls the video device 160 to output video stimuli to the assessed individual. In various aspects, the stimuli can include various videos selected by the practitioner or via a program, which may be silent or include audio. These videos may be prerecorded for playback to the assessed individual, or may be controlled in the output via the practitioner (e.g., similar to a video game). Additionally or alternatively, the video device 160 may provide some or all stimuli as audio-only stimuli.

At block 530, the various sensors collect and process reactions from the assessed individual to the stimuli provided in block 520. Various algorithms, programs, and services may be used to identify changes in heart rate, emotional state, attention, or combinations thereof based on the inputs from various sensors and recording devices, and the time of these changes relative to the provided stimuli.

At block 540, a machine learning algorithm uses the reactions collected and processed per block 530 to generate an assessment of the information processing capabilities of the assessed individual. In various embodiments, the machine learning algorithm bases the assessment on the age of the assessed individual and what is an age-appropriate reaction for a neurotypical person of the same age or a typical reaction for a person of the same age having a cognitive disability of a given type to help the practitioner diagnose whether the assessed individual exhibits one or more cognitive disabilities that affect information processing ability.

FIG. 6 is an example computing device 600, as may be used with a testing booth 100, according to aspects of the present disclosure. In various embodiments, the divider 150 include a computing device 600 in communication with the video display device and the video recording device of an analysis device 160. The computing device 600 may include at least one processor 610, a memory 620, and a communication interface 630 and may be used as a controller for the system described herein.

The processor 610 may be any processing unit capable of performing the operations and procedures described in the present disclosure. In various embodiments, the processor 610 can represent a single processor, multiple processors, a processor with multiple cores, and combinations thereof.

The memory 620 is an apparatus that may be either volatile or non-volatile memory and may include RAM, flash, cache, disk drives, and other computer readable memory storage devices. Although shown as a single entity, the memory 620 may be divided into different memory storage elements such as RAM and one or more hard disk drives. As used herein, the memory 620 is an example of a device that includes computer-readable storage media and is not to be interpreted as transmission media or signals per se.

As shown, the memory 620 includes various instructions that are executable by the processor 610 to provide an operating system 622 to manage various features of the computing device 600 and one or more programs 624 to provide various functionalities to users of the computing device 600, which include one or more of the features and functionalities described in the present disclosure. One of ordinary skill in the relevant art will recognize that different approaches can be taken in selecting or designing a program 624 to perform the operations described herein, including choice of programming language, the operating system 622 used by the computing device 600, and the architecture of the processor 610 and memory 620. Accordingly, the person of ordinary skill in the relevant art will be able to select or design an appropriate program 624 based on the details provided in the present disclosure.

Additionally, the memory 620 can include one or more machine learning algorithms 626 or models for aiding a medical practitioner in assessing an information processing capability of an assessed individual who is recorded (via the video recording device) reacting to video displayed by the video display device.

The programs 624 and the machine learning algorithms 626 are used for diagnosing intellectual disabilities, particularly in children and newborns, and provide an automated assessment tool powered with machine learning capable of assessing the information processing capabilities of various assessed individuals in a few minutes. The t programs 624 and the machine learning algorithms 626 output a stimulus (e.g., a video) to the assessed individual, and collect heart rate data (e.g., via heart rate monitoring equipment), facial expression (e.g., facial expression recognition algorithms), gaze direction (e.g., via video recording equipment) as physiological and behavioral data to compare against the stimulus.

The information processing procedure assesses the assessed individual's ability to create mental representations for complex visual and auditory sequences at appropriate age rates for that individual (e.g., whether an N month-old reacts as an N-month old child is expected to react). The machine learning algorithms 626 measure the speed of information processing, a dimension that can distinguish children across the first three years of life. The machine learning algorithms 626 does not require expressive language, object manipulation, or compliance with the requests of the examiners and therefore circumvents the confounded measures inherent in conventional tests of mental development. Thus, when used in combination with conventional tests, which almost invariably reveal delayed development among children with autism, a differential diagnosis can be made distinguishing delayed cognitive development from delayed expressive development. The present disclosure thereby allows for a more precise assessment of mental ability and more appropriate and effective intervention strategies.

The present disclosure possesses numerous benefits and advantages over the traditional assessment tools of diagnosing intellectual disabilities in assessed individuals such as the Bayley and IQ tests which were both shown to be inaccurate and with a large error margin. The traditional assessments of mental development are standardized tests that compare the performance of an assessed individual to a much larger and normally distributed population of assessed individuals of the same age. These conventional assessments do not separate mental capacity from the development of the main means of communicating that ability, namely expressive language, and the use of practical and symbolic objects, which is of particular concern in assessed individuals who are not verbal. At the early ages, virtually all these assessment procedures require the use of functional objects, expressive language, and compliance with the requirements of the examiner to deduce mental capacity. There are additional problems in traditional testing of infants' mental development, as these traditional tests have no predictive validity during the first approximately 1.5 years of life. These limitations in the traditional assessment methods result in children being identified as mentally retarded when they may not be.

The measures used to infer mental ability in traditional tests are confounded with the defining features of the disability. For example, in the case of autism spectrum disorders, which are defined by delays in the use of objects, delayed expressive language, and behavioral and social difficulties that reduce the likelihood of compliance with the requests of the examiner, these profiles can only negatively affect the child's score in traditional tests and do not provide an unbiased measure of mental processing.

This perplexing situation has implications for both autism etiology and intervention strategies. As the child grows older, expressive language plays a dominant role in intelligence testing and significantly contributes to further cognitive development.

The occurrence estimates of mental retardation in children with autism spectrum disorder range with a median of 65%, and most children with autism spectrum disorder have co-occurring mental retardation. Using the described approach, 75% of cases in which children displayed delayed assessments on traditional tests and age-appropriate information processing were able to overcome their delays on traditional tests following appropriate intervention. Because there is no known cure for mental retardation, such an assessment can severely negatively impact the prognosis for a child with autism reducing hopes and expectations for the child. A misdiagnosis with mental retardation may prevent the child from receiving proper treatment and prevent physicians and psychologists from intervening. As a result, this would severely affect their mental development leading them to acquire mental retardation.

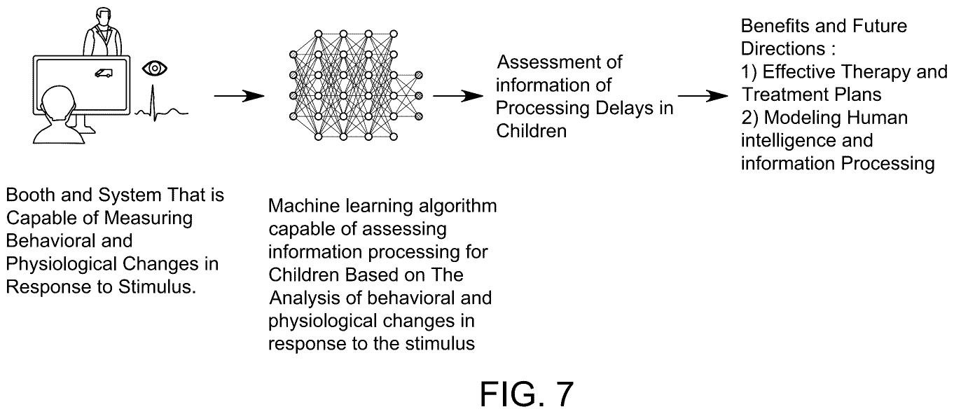

Accordingly, the present disclosure may be used not only as a diagnosis tool for medical professional, but as a prophylaxis for providing care to avoid exacerbating or causing mental retardation due to false positives in individuals assessed under the traditional tests. Operation of the programs 624 and the machine learning algorithms 626 may be understood with reference to FIG. 7.

The communication interface 630 facilitates communications between the computing device 600 and other devices, which may also be computing devices 600 as described in relation to FIG. 6. In various embodiments, the communication interface 630 includes antennas for wireless communications and various wired communication ports. The computing device 600 may also include or be in communication, via the communication interface 630, one or more input devices (e.g., a keyboard, mouse, pen, touch input device, etc.) and one or more output devices (e.g., a display, speakers, a printer, etc.).

Accordingly, the computing device 600 is an example of a system that includes a processor 610 and a memory 620 that includes instructions that (when executed by the processor 610) perform various embodiments of the present disclosure. Similarly, the memory 620 is an apparatus that includes instructions that when executed by a processor 610 perform various embodiments of the present disclosure.

Although the present invention has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive. It will be evident to the annotator skilled in the art to freely combine several or all of the embodiments discussed here as deemed suitable for a specific application of the invention. Throughout this disclosure, terms like “advantageous”, “exemplary” or “preferred” indicate elements or dimensions which are particularly suitable (but not essential) to the invention or an embodiment thereof, and may be modified wherever deemed suitable by the skilled annotator, except where expressly required. Accordingly, the scope of the invention should be determined not by the embodiments illustrated, but by the appended claims and their equivalents.

1. A device, comprising:
a base panel, having a trapezoidal surface defined in a first plane with a front edge parallel to a back edge, a first side edge disposed at a first angle between the front edge and the back edge, and a second side edge disposed at an inverse of the first angle between the front edge and the back edge;
a first side panel attached to the first side edge of the base panel, extending perpendicularly to the first plane;
a second side panel attached to the second side edge of the base panel, extending perpendicularly to the first plane;
a roof panel, connected between the first side panel and the second side panel in a second plane that intersects the first plane; and
a divider, connected between the first side panel, the second side panel, the roof panel, and the base panel, extending perpendicularly from the base panel and including a video display device and a video recording device.

2. The device of claim 1, further comprising:
a first front panel rotatably connected to the first side panel; and a second front panel rotatably connected to the second side panel.

3. The device of claim 1, wherein the second plane intersects the first plane at the first angle.

4. The device of claim 1, wherein the video display device and the video recording device are included on first side of the divider, and the device further comprises:
a control device included on a second side of the divider, opposite to the first side.

5. The device of claim 1, further comprising:
a computing device included within the divider in communication with the video display device and the video recording device, the computing device including a processor and a memory that includes instructions that when executed by the processor provide a machine learning algorithm to assess an information processing capability of an assessed individual recorded reacting to video displayed by the video display device via the video recording device.

6. The device of claim 1, wherein the base panel is positioned at a predefined distance from a floor that the first side panel and the second side panel rest on.

7. The device of claim 1, wherein the divider is positioned at a predefined distance away from the front edge.

8. The device of claim 1, wherein the divider is positioned at a predefined distance away from the back edge.

9. The device of claim 1, wherein a height of the first side panel and the second side panel is less than 1.8 meters.

10. The device of claim 1, wherein inner surfaces of the first side panel, the second side panel, the roof panel, and the base panel define a viewing funnel and include sound proofing.
